(19) United States
(12) Patent Application Publication
(10) Pub. No.: US 2005/0158402 A1
(43) Pub. Date: Jul. 21, 2005

(54) Preparation of Chinese herbal composite recipe used in environmental sanitation

(76) Inventor: Chien-Yung Lee, Kaohsiung Hsien (TW)

Correspondence Address: BACON & THOMAS, PLLC, 625 SLATERS LANE, FOURTH FLOOR, ALEXANDRIA, VA, 22314, US

(21) Appl. No.: 10/858,055

(22) Filed: Jun. 2, 2004

(30) Foreign Application Priority Data
Jan. 20, 2004 (CN) 200410002767.4

(51) Int. Cl. A61K 35/78

(52) U.S. Cl. 424/725

(57) ABSTRACT

A Chinese herbal composition for environmental sanitation is free of toxin and user-friendly water-soluble spray. The practical applications of this spray include pest annihilation, better environmental sanitation, germicide and viruses inhibitive functions. It is also effective in prevention against epidemic and prevalent disease. This composition is composed of Akebia trifoliata, Polygonum perifoliatum, Euodia rutaecarpa, Stemona tuberose, Bryophyllum pinnatum, and Sophora flavescens. These drugs have passed the western and oriental pharmacologists' pharmacological experiments and proved of pesticide, germicide and viruses inhibitive effects. As based on above, the inventor also concerns the multiple effects of drug composition. By using revolutionary extraction and scientific preparation methods, the non-toxic Chinese herbal ingredients can be transformed into potent annihilative preparation.

### BACKGROUND OF THE INVENTION

1. Field of the Invention

The present invention relates generally to medicine, and more particularly to a Chinese herbal composition used in environmental sanitation.

2. Description of the Related Art

To repel flies, gnat or mosquitoes, people used to burn the primitive burning grass for smoke; afterwards, they used to burn Acorus calamus, Euodia rutaecarpa, Atratylodis rhizona, Sandal wood, or sprayed realgar wine. There are some delicate actions like burning incenses and carrying pack of herbs for repelling the pests in ancient China. It is obvious that the ideas and solutions of environmental sanitation have been developed from ancient time. Following the progress of era, human have had the needs for simpleness and effectiveness since the twentieth century, thereby making the evolution of sprays, coils, electric coils and many other types of pest control preparations commercially available for annihilation mosquitoes, repelling flies, and eradiating cockroaches. Although they belong to the products of science, they are not fully confirmed to be effective. Something in common is that they are poisonous. During the period of SARS (severe acute respiratory syndrome), many patients and medical crews died for the closed disinfection method mistakenly used in one province of China.

The environmental sanitary products in the market have single or limited usage.

Products which have germicide and viruses inhibition were paid special attentions by the authorities as things became serious. They are expensive and hard to purchase in usual time. Many people even do not know how to use them.

The environmental sanitary sprays mostly contain deadly poisons but the manufacturer labeled them with poisonous and cautions only. They don't care or ignore the consequences of the remaining poisons. Since the present inventor is a cancer researcher, in many of his cases, he has found many direct connections between the usage of this kind of pesticide and the issue of cancers. Contacting mucosa by nose and lungs will cause the defense cells to mutate into caner cells.

Does it necessary for drugs to possess poisons to kill pests and eradiate bacteria and inhibit viruses? This idea is not in accordance with the facts. You can find so many non-toxic plants or ingredients from the books of Chinese medicine but still have the effects of pesticide, germicide and viruses inhibition. Especially in the 1950s, under the projects of policies and efforts by the experts, completion of experiments of many drugs and the analysis of effective ingredients have been done for the Chinese medicine. All of these contributions give the Chinese herbal medicine the opportunities of further development. How to use the information contributed by the modem experts and obey the theories of Chinese medicine to push non-toxic, complicated ingredients, less potent, and less pesticidally effective Chinese herbs to exceed poisonous agents in the contemporary worlds? It was only a wonderful dream before, but now this dream will come true.

### SUMMARY OF THE INVENTION

The primary objective of the present invention is to provide a Chinese herbal composition, which employs revolutionary extract methods in common applications of environmental sanitation to kill or to repel the common pests like mosquitoes, flies, cockroaches, lice, fleas and mites. It also includes the effects to eradiate many bacteria, fungus and viruses inhibition and possess the objective to prevent epidemic and confluent diseases.

The secondary objective of the present invention is to employ the fact recited below to break the arbitrarily mode of “annihilation pests and bacteria requires poison”, and to develop the application values of using mild and gentle Chinese medicine. The present invention can replace poisonous chemicals and maintain human safety and health

To achieve the foregoing objectives, I supply the theories and applications, drug dispensing, drug preparation method, pharmacology description, range and method of application, caution, and detail drug description.

### DETAILED DESCRIPTION OF PREFERRED EMBODIMENTS

The entities of the present invention are based on the implementation of “original theories and its revolution” from Chinese medicine. The applicable features of its contents include “pharmacology experiments of modem Chinese herbs” and “the right idea on environ of target creatures exist”. I use them to make the “requirements of annihilation” and develop the evolutional extract form and scientific methods of preparation. The completion of the present invention depends on such delicate steps and I will specify them below.

Scientific researches about bacteria and viruses, no matter modem or the western type, all trends to the retrograde searching of their real form and source of parasite. The Chinese medicine is focused on the environmental factors and the effects to human bodies. The forms of them are different but the goals are the same. The books of Chinese medicine contain many methods of dealing with bacteria and viruses. The methods have been being used since the ancient time. The bacteria and viruses were named by the six evils of as wind, cols, summer heat, dampness, dryness, fire and their combination. “Worms are hatched from dampness and heat” is the common statement of Chinese medicine. The Chinese always put clothes, quivers and groceries under sunshine after the rainy season. The purpose of doing this is to evaporate dampness and annihilation so-called yin-evils by sunshine. The so-called yin-evil is the bacteria we know. However, many people do not understand such things to hardly believe them. In light of this, an innovative statement was proposed, reciting that “Every creature is composed by six elements such as energy, space, substance, dampness, airflow, and temperature”. The bacteria and viruses belong to creatures and are composed by the six elements and will be dead without any of the six elements. The cooperation of dampness and temperature can generate gases from spaces in the substance, such gases including tiny substance, which is the so-called bacteria and viruses harmful to the living creatures. Depend on the proportions of the six elements, there are thousands even more bacteria and viruses had been category and named or not. Therefore, I said from the pest and bacteria annihilation and viruses inhibition physiology of Chinese medicine, the point of this strategy is the content of six elements and changes of environ. In other words, by choosing adequate drugs and using their effective ingredients can promote transformation of worms, bacteria and viruses' six elements immediately and change the status of the environment they exist. The goal of annihilation is achieved no matter toxic or non-toxic.

The Chinese medicine is the masterpiece that has collected a great number of medical experiences of thousands of years. Based on values of the Chinese medicine and to bring connection with the modem science, the Chinese government has been devoted to the pharmacological experiments of Chinese medicine and the arrangement and compilation of related knowledge. The achievement of the above works is very remarkable. It offers the precious information and the convenience of applications to bio-technique researchers. In the present invention, I adopt the analysis about the ingredients and results and data of the aforementioned pharmacological experiments. In the situation of understanding the effective ingredients, they can be well preserved and utilized during the process of preparation. Since the pharmacological experiments are quite credible, I am sure that they can make up for the deficiency of research instruments and meet the requirements of the invention. For example, the decoct of Buodia rutaecarpa shows stronger inhibiting effects on V. cholerae. It also shows certain degree of inhibiting effects on Pseudomonas aeruginosa, Staphylococcus aureaus and some common disease-causing fungus. (W-F, Dam etc. Chinese medicine 1952, 38(4):315) The evodiamine shows anti-viruses effect on rats, which were infected by Columbia S K strand of viruses. (Chou S C. CA, 1967, 67:61622j)

The management of oral medicine for curing disease and topical usage to kill microbe for the environment are quite different. There must have clear idea to realize the situation of pests, lice, bacteria and viruses hosted or spread in the environment. Then, we can kill or repel them reasonably. The pests, lice, bacteria and viruses have the same existing condition in the environment. They must have comfortable shields and they will disappear as we place these shields under strong streams or sunshine.

In addition, the pests mainly on the damp ground which are dark and less ventilated corners are subject to reproduction. The bacteria mostly attach themselves to something under shadow after leaving their hosts. The viruses re-disperse into the air from the hosts, too. As the size of the creatures is concerned, the worm and lice are the biggest one, bacterium is the next biggest, and virus is the least one. However, as regards the capabilities of spreading themselves in the environment are contrary to the size. The worm and lice are the least, bacterium is the next, and virus is the greatest. Tiny bacteria, especially viruses, are always circulating between the shields with air. It must have something extremely tiny and dense material, then perform close contact with it. From this point, we can recall the density of Chinese incense and its volatile substance. However, the incense has limited bactericidal effects and it tends to hurt human bodies like the toxic agents.

The eradication of pests and microbes in the environment has some criteria. Except carefully selecting, composing, dispensing, and testing drugs, we have to analyze these criteria. Because the targets are tiny, there must be some tinier drugs for contacting them and having sufficient penetrability after the contact to transform the targets' inside (six elements) immediately. Drugs possess such effects are qualified for the criteria. In addition, it must change the atmosphere in the range of the shield to elongate and enhance the potency of drugs and let it not suitable for the targets to reproduce.

For the reasons as stated above, the present invention is through the composition of individually potency-tested and potency-multiplied drugs and the process of complicated extraction to be micronized for abstracting many oily essence that can be emulsified to reduce the surface and interface tensions, and then transform it into water soluble emulsion. This make it become the most tiny and penetratable preparation. This new kind of annihilation method by emulsion is the mechanism to enhance the potency. The result of volatile soluble in water can elongate time of evaporation. It can perform some lasting atmosphere, good to human and animals but not for worm, lice, bacteria and viruses in the shielded range of target

The Chinese composition spray agent in according with the present invention for environmental sanitation is prepared by Chinese herbs listed below, and then I concentrate the extraction and make powder by ratio with specific ratio of percolate. I pulverize them by grinding and sieving with emulsified agents added.

(1) Concentrated Chinese herbal powder and its ratio:

| | | |
|---|---|---|
| Akebia trifoliate | 80-120 | gm |
| Stemona tuberose | 80-120 | gm |
| Polygonum perfoliatum | 80-120 | gm |
| Bryophyllum pinnatum | 80-120 | gm |
| Euodia rutaecarpa | 60-100 | gm |

| | |
|---|---|
| Sophora flavescens | pulverized as adjuvants. |

(2) Percolate and its ratio:

| | |
|---|---|
| distilled water | 60-80% |
| glycerin | 8-12% |
| alcohol | 12-24% |

(3) Emulsified agents used in original solution Of 3000 ml:

| | | |
|---|---|---|
| Gum Arabic | 30-50 | gm. |
| Perilla frutescens oil | 30-45 | ml. |
| Pogostemon cablin oil | 120-35 | ml. |
| Sandalwood oil | 15-25 | ml |

There are three steps recited below for preparation of the present invention.

(1) Take concentrated Chinese composition powder 600 gm and percolate as well. For the first time, douse the powder in the percolate of 1200 ml then pass it to a golden mill for 2-3 times, douse for 2 hours, and leach the first percolate. For the second time, douse the same powder in the percolate of 1000 ml for 2 hours, and then leach the second percolate. For the third time, douse the power in distilled water of 1000 ml for 4 hours and then leach the third percolate. Thus, there are totally three percolates for later use.

(2) Take gum Arabic, Perilla frutescens oil, Pogostemon cablin oil and Sandalwood oil to make emulsion. Blend the emulsion into the percolate of the step (1) and let it through the golden mill for three times to get a percolate.

(3) Blend Mirabilite, benzoic acid, and other additives into the percolate of the step (2). Stir the admixture evenly and then let it through the golden mill for 2-3 times to get the agent of the present invention.

The pharmacological specification is recited below.

1. Akebia trifoliate: The Akebia trifoliate shows inhibitive effects on Gram's positive and Gram's negative bacteria such as Shigella and Salmonella typhi in vitro. It contains glycosides and saponins which can produce multiple effects.

2. Polygonum perfoliatum: The Polygonum perifoliatum shows the anti-virus potency against Asian A-type influenza viruses and para-influenza I-typed viruses. The decoctant shows anti-bacterial potency against Shigella, Smith S, flexneri and S. sonnei. The decoctant show stronger anti-bacteria effects on Haemophilus influenzae, Salmonella typhi, Corynebacterium diphtheriae, Bacillus anthracis, E. Coli and β-streptococcus.

3. Euodia rutaecarpa: It contains many alkaloids and has the annihilative effects on worms and leeches. The evodiamine shows anti-virus effect on rats which are infected by Columbia SK viruses. It shows stronger inhibitive effects on Vibrio cholerae in vrtrohas certain degree of inhibitive effects on Pseudomonas aeruginosa, Staphylococcus aureaus and some common disease-causing fungus.

4. Stemona tuberose: It contains 13 alkaloids and 6 organic acids. It can kill body lice, pubic lice, maggots, bed bugs and many other pests. It is an apparently pesticide which can make eggs of lice and pests hard to hatch and show effects on anti-microbes.

5. Bryophyllum pinnatum: It contains many organic acids, sugar glycosides, steroids and ketones. The juice from leaves of Bryophyllum pinnatum in 5% (v/v) concentration shows broad spectrum of bactericidal effects on Gram's positive and Gram's negative bacteria. It includes many resistant bacteria separated from clinical.

6. Perilla frutescens: The decoctant of Perilla frutescens shows inhibitive effect on ECHO11 viruses in vitro. The perillaldehyde and tadeonal show apparently cooperative anti-fungal effects. The oil from leaves of Perilla frutescens shows apparently inhibitive effects on nature pollution sources of Aspergillus niger, Penicillium and yeasts.

7. Pogostemon cablin: It contains pathchoulo alcohol, pathchoulo diol and many alkene volatile oil. It shows apparently inhibitive effects on fungi such as C. albicans, Cryptococcus neoformans and Rhizopus stolonifer. It shows certain degree of inhibitive effect on type A hemolytic Streptococcus.

8. Santalum album: The main ingredients are sesquiterpene compounds such as α-santol and β-santol up to 90% or above. It has stronger anti-bacteria effects and also has inhibitive effect on Shigella species and M. avium.

The range and method of application for the present invention is recited below.

1. The present invention is a water soluble spray agent, having certain degree of pest annihilative, germicidal, and virus-inhibitive effects.
A. In pesticide: It is pesticidal to mosquito, fly, cock roach, flea, wood lice, mite and head lice, body lice, pubic lice, dog lice and eggs which located on human body surface.
B. In germicide: It is germicidal to influenza, cephalitis, hepatitis, diphtheria, dysentery, cholera and anthrax bacteria vice versa. Especially Gram's positive and Gram's negative bacteria with nature causing Aspergillus niger and Penicillium.
C. In viruses: It is inhibitive to Asian A-type influenza viruses and para-influenza type I viruses, Columbia SK viruses, Echo viruses and Enterovirus.

2. It is available to manually spray the present invention in kitchen, living room, and other rooms, especially at the moist and bad ventilated corners or the places those pests gathered. It is also available to spray the present invention directly to where body lice, pubic lice, head lice or dog lice is hosted.

3. The present invention can be sprayed by power sprayer on the inner corners of buildings, walls, poles and utilities or dark damp corners at hospital, clinic and public areas or in the periods of epidemic bacterial or viral infection.

There are some cautions for using the present invention.

1. The spray prepared by the present invention is harmless to animals and human. It does not cause any stimulation as contact with skin, eyes, nose, mouth and tongue. It will all right if a little amount of the spray is eaten. Human and animals can breathe normally and stay in closed space during the period of ejecting the spray.

2. The present invention is a topically Chinese medicine pesticide, germicide and virus-inhibitive agent. It is prohibited to take it orally. It may cause gastrointestinal symptoms when taken orally. People who mistakes it can mix rare green bean powder, rare glycyrrhizin powder and talcum powder 6 gm of 3-6 gm respectively with cold water and take it orally, and thus they may feel laxative and then better.

3. The present invention should be stored in cool and dry places to prevent deterioration caused by high temperature.

The detailed pharmacological specification of the Chinese herbs indicated above is recited below.

NO: 1

| | | | |
|---|---|---|---|
| HERBAL NAME | Mutong | OFFICIAL NAME | Akebia trifoliate |
| | | | (Thunb.) |

| | |
|---|---|
| REFERENCE BASIS | China Pharmacopoeia Volume III pp 329 (China Sheng-Ghai |
| | scientific technology publication) |
| GROWING DISTRICT | Akebia trifoliate grows mainly in Si-chuan. Collect some old |
| & COLLECT SEASON | vines in autumn and winter. Dry or bakE then slice them into |
| & PARTS | thin pieces. |
| PREPARATION & | Take 12 kg of Akebia trifoliate and soak in water for 3 hours. |
| EXTRACTION | Collect percolate for 2 times then evaporate the residue |
| PROCESS & METHOD | water to get 5 liters of fluid extract. Adding 2 kg of Sophora |
| | flavescens powder and stir well. Drying it in 60° C. then |
| | pulverized by passing #120 sieve to make powder. Collect |
| | and store them for later use. |
| EFFECTIVE | The vines of Akebia trifoliate contain betulin, oleanic acid, |
| INGREDIENT | hederagenin, akebin. It also contains stigmasterol, β- |
| | sitisterol, daucosterol, inositol, sugar and sodium salts. |
| ORIGINAL | The extract of Akebia trifoliate (1:20) shows inhibitive |
| RECORDED | effects on Gram's positive and Gram's negative bacteria |
| PHARMACOLOGICAL | such as Shigella and Salmonella typhi in vitro. (Wang-yu et |
| EFFECTS | al. Plant report, 1953, 2(2): 312) |

NO: 2

| | | | |
|---|---|---|---|
| HERBAL NAME | Gangvangai | OFFICIAL NAME | Polygonum |
| | | | perifoliatum L. |

| | |
|---|---|
| GROWING DISTRICT | It grows in southeast provinces of China, Fu-jann, and |
| & COLLECT SEASON | Taiwan. Collect them in full blossom in summer and |
| & PARTS | autumn. Cut the upper parts of plants and drying. Bundle |
| | them as half drying and continue drying under shades. |
| PREPARATION & | Take 12 kg of Polygonum perifoliatum and soak in water for |
| EXTRACTION | 3 hours. Collect percolate for 2 times then evaporate the |
| PROCESS & METHOD | residue water to get 5 liters of fluid extract. Adding 2 kg of |
| | Sophora flavescens powder and stir well. Drying it in 45° C. |
| | then pulverized by passing #120 sieve to make powder. |
| | Collect and store them for later use. |
| EFFECTIVE | The all plant contains kaemferol, methylcaffeate ester, |
| INGREDIENT | quercitin, caffeic acid, protocate chuic acid, methyl caffinate |
| | ester, p-coumaric acid, feruic acid, vanillic acid, arbutinic |
| | acid and betulinic acid. It also contains sterol fatty acid |
| | esters, plant steroid β -d-glucoside, 3,3,4,4,-tetramethyl |
| | ellagic acid, 3,3-dinethyl ellagic acid, racemic dimethyl |
| | tartrate ester and long chain fatty acids. There are indicant |
| | and tannic acids. |
| ORIGINAL | The viruses test out of chicken embryo shows the decoctant |
| RECORDED | of Polygonum perifoliatum show the anti-viruses potency |
| PHARMACOLOGICAL | against Asian type A influenza viruses and para-influenza I |
| EFFECTS | viruses are 1:160 and 1:640, respectively. (J-Y, Ma etc. |
| | Shan-si new medicine, 1979, 8(12): 42) |
| | The anti-bacterial potency of the decoctant to Shigella, |
| | Smith S, flexneri and S. sonnei are 1:512, 1:128, 1:64 and |
| | 1:15, respectively. (C-1, Lee, Fu-chien Chinese medicine, |
| | 1960,5(7): 310) The decoctant show stronger anti-bacteria |
| | effects on Staphylococcus aureaus, β -streptococcus, |
| | Bacillus anthracis, Corynebacterium diphtheriae, Bacillus |
| | subtilis, E. Coli, Salmonella typhi, Pseudomonas aeruginosa |
| | and Haemophilus influenzae. (J-Y, Ma etc. Shan-si new |
| | medicine, 1979, 8(12): 42. National Chinese medicine |
| | pharmacopeia Vol I, edition 1, 1976, 416) |

NO: 3

| | | | |
|---|---|---|---|
| HERBAL NAME | Wuzhyu | OFFICIAL NAME | Buodia rutaecarpa |
| | | | (juss.) |

| | |
|---|---|
| REFERENCE BASIS | China Pharmacopoeia Volume IV pp 927 (China Sheng-Ghai |
| | scientific technology publication) |
| GROWING DISTRICT | It grows in Qui-zhou and Kwang-si mainly. Chang-de of |
| & COLLECT SEASON | Hu-nan has the best quality. Collect them as the fruits show |
| & PARTS | tea-green color and the carpel do not separates. You must |
| | take all spikes of fruits before the drops of dew dry. Dry |
| | them and remove the handles. |
| PREPARATION & | Take 12 kg of Buodia rutaecarpa and soak in water for 3 |
| EXTRACTION | hours. Collect percolate for 2 times then evaporate the |
| PROCESS & METHOD | residue water to get 5 liters of fluid extract. Adding 2 kg |
| | Sophora flavescens of powder and stir well. Drying it in 45° |
| | C. then pulverized by passing #120 sieve to make powder. |
| | Collect and store them for later use. |
| EFFECTIVE | The seeds contain cis-5,8-decadieneoic acid. The volatile oil |
| INGREDIENT | has evodene, evodol and limonin. The fruits contain many |
| | alkaloids such as evodiamine, rutaecarpine, alkenes, ketones |
| | and aminoesers. Otherwise, it also contains 8 amino acids |
| | including aspartic acid. |
| ORIGINAL | The decoctant of Buodia rutaecarpa show stronger inhibitive |
| RECORDED | effects on V. choleroae. It also shows certain degree of |
| PHARMACOLOGICAL | inhibitive effects on Pseudomonas aeruginosa, |
| EFFECTS | Staphylococcus aureaus and some common disease-causing |
| | fungus. (W-F, Dam etc. Chinese medicine 1952, 38(4): 315) |
| | The evodiamine show anti-viruses effect on rats which |
| | infected by Columbia SK viruses. (Chou SC. CA, 1967, |
| | 67: 61622j) The water, ethanol and ether decoctants of |
| | Buodia rutaecarpa can kill pig ascarids, earth worms and |
| | leeches in vitro. (Y-R, Wu etc. Chinese medicine, 1948, |
| | 34(10): 437) |

NO: 4

| | | | |
|---|---|---|---|
| HERBAL NAME | Baibu | OFFICIAL NAME | Stemona tuberose |
| | | | Lour |

| | |
|---|---|
| REFERENCE BASIS | China Pharmacopoeia Volume VIII pp 189 (China |
| | Sheng-Ghai scientific technology publication) |
| GROWING DISTRICT | Stemona tuberose grows in southeast province of China, |
| & COLLECT SEASON | Hu-nan, Hu-peh and Si-chuan. Collect them after 3 years of |
| & PARTS | planting. Remove tubers after wilting of upper parts of shoot |
| | before spring. Remove roots and boiling in hot water to well |
| | done then drying or baking them. |
| PREPARATION & | Take 12 kg of Sophora flavescens and soak in water for 3 |
| EXTRACTION | hours. Collect percolate for 2 times then evaporate the |
| PROCESS & METHOD | residue water to get 5 liters of fluid extract. Adding 2 kg |
| | Dioscorea collettii of powder and stir well. Drying it in 60° C. |
| | then pulverized by passing #120 sieve to make powder. |
| | Collect and store them for later use. |
| EFFECTIVE | The Stemona tuberose contains 13 alkaloids including |
| INGREDIENT | syemonine, tuberostemonine and isotuberostemonine. It also |
| | contains sugar, fats, proteins, ash and 6 different acids |
| | including oxalic acid. |
| ORIGINAL | 1. Anti-microbe. |
| RECORDED | 2. Anti-parasite: In vitro tests show that 50% solution can |
| PHARMACOLOGICAL | kill pin worm in 20 hours. The tuberostemonine can paralyze |
| EFFECTS | Ascarid. |
| | 3. Insect annihilation effects: The Stemona tuberose water |
| | solution and alcohol solution has effects on lice and cause |
| | eggs of lice not hatch. It also show contact annihilation |
| | effect on 10 or more pests including maggot, larva of |
| | mosquito, bed bug, aphids, larva of Chilo simples and spider. |

NO: 5

| | | | |
|---|---|---|---|
| HERBAL NAME | Luodishenggen | OFFICIAL NAME | Bryophyllum |
| | | | pinnatum (L. f.) |

| | |
|---|---|
| REFERENCE BASIS | China Pharmacopoeia Volume III pp 752 (China Sheng-Ghai |
| | scientific technology publication) |
| GROWING DISTRICT | It grows in Fu-jann, Taiwan, Kwang-tong, Kwang-si and |
| & COLLECT SEASON | Yun-nan. |
| & PARTS | |
| PREPARATION & | Take 36 kg of fresh leaves and stems of Bryophyllum |
| EXTRACTION | pinnatum and put them into colloid mill for grinding. Collect |
| PROCESS & METHOD | percolate for 3 times and place it on the steel plates them |
| | transfer to “low temperature gas chilled dryer” then |
| | evaporate the residue water to get 5 liters of concentrate |
| | solution. Adding 2 kg of Sophora flavescens powder and stir |
| | well. Drying it in low temperature then pulverized by |
| | passing #120 sieves to make powder. Collect and store them |
| | for later use. |
| EFFECTIVE | The Bryophyllum pinnatum contains cis-aconitic acid, |
| INGREDIENT | ascorbic acid, eugenic acid, caffeic acid, p-hydroxy benzoic |
| | acids and many organic acids. It also contains quercitin, |
| | kafmferol, avicularin, 18 α - oleanane , ψ -taraxasterol, |
| | β-Amyrin acetate, 24-ethyl-25 hydroxy cholesterol, α- |
| | Amyrin, β-Amyrin, Bryophyllum pinnatum steroids, |
| | ketones, alkene ketones and alcohols. |
| ORIGINAL | The juice from leaves of Bryophyllum pinnatum in 5%(v/v0) |
| RECORDED | concentration show broad spectrum of bactericidal effects on |
| PHARMACOLOGICAL | Gram's positive and Gram's negative bacteria such as |
| EFFECTS | Staphyllococcus aureaus, Streptococcus pyrogens, S. |
| | faecalis, E. Coli, Proteus, Klebsiells, Shigella, Salmonella, |
| | Serratia marcescens and Pseudomonas aeruginosa. They |
| | including many resistant bacteria separated from clinical. |
| | (Obaselki E. Afr J Med Sci, 1985, 14(3-4); 199) |

NO: 6

| | | | |
|---|---|---|---|
| HERBAL NAME | Kushen | OFFICIAL NAME | Sophora |
| | | | flavescens |
| | | | Ait |

| | |
|---|---|
| REFERENCE BASIS | China Pharmacopoeia Volume IV pp 634 (China Sheng-Ghai |
| | scientific technology publication) |
| GROWING DISTRICT | Grow in everywhere in China. Take all plants in September |
| & COLLECT SEASON | to October and remove the upper part, base of stem and |
| & PARTS | fibrous roots. Cut fibrous roots into single strand. Wash out |
| | dirt then dry or bake it. |
| PREPARATION & | Take 12 kg of Sophora flavescens and soak in water for 3 |
| EXTRACTION | hours. Collect percolate for 2 times then evaporate the |
| PROCESS & METHOD | residue water to get 5 liters of fluid extract. Adding 2 kg |
| | Dioscorea collettii of powder and stir well. Drying it in 60° C. |
| | then pulverized by passing #120 sieve to make powder. |
| | Collect and store them for later use. |
| EFFECTIVE | The roots contain 13 alkaloids including matrine and many |
| INGREDIENT | flavonoids mixtures. Otherwise, the roots contain tri-terpene |
| | saponin, soy saponin and kurarinone. |
| ORIGINAL | The in vitro experiments show that 1% matrine has apparent |
| RECORDED | bacteria inhibitive effects on Shigella, E. Coli, Proteus, |
| PHARMACOLOGICAL | B-streptococcus and Staphylococcus aureaus. The total |
| EFFECTS | flavones of Sophora flavescens and their ethanol extract |
| | have annihilation or growth inhibitive effects on Giardia |
| | lamblia Stiles in vitro. |
| PHARMACOLOGICAL | It can use specific alkaloids and saponins to achieve goals of |
| APPLICATIONS, | annihilation pests and bacteria. The inventor observed 20 |
| PURPOSES | concentrated Chinese medicine drugs powder all using |
| | starch as adjuvant for 20 years and found that the Sophora |
| | flavescens did not eat into by worms and not change its |
| | quality. The package still remains intact. It shows the effect |
| | of germicide. |

NO: 7

| | | | |
|---|---|---|---|
| NAME | Zisuye | OFFICIAL NAME | Perilla frutescens |
| | | | (L.) Britt. |

| | |
|---|---|
| REFERENCE BASIS | China Pharmacopoeia Volume VII pp 115 (China |
| | Sheng-Ghai scientific technology publication) |
| GROWING DISTRICT | Perilla frutescens grows best in Kwang-tong, Kwang-si and |
| & COLLECT SEASON | Hu-peh. Collect them in July or August as leaves and twigs |
| & PARTS | prosper. Remove leaves after spread on the ground or place |
| | in well ventilated areas for drying. |
| PREPARATION & | Take 12 kg leaves of Perilla frutescens and pressed firmly in |
| EXTRACTION | the distillation flask. By using distillation method to get |
| PROCESS & METHOD | volatile oil. Collect and store them for later use. |
| EFFECTIVE | The Perilla frutescens contains perillaldehyde, limonene, β- |
| INGREDIENT | caryophllene, and linalool. It also contains perilla-alcohol- |
| | β -d-pyranoglucoside. |
| ORIGINAL | The distilled fractions of Perilla frutescens have broad |
| RECORDED | spectrum of anti-bacteria effects. The main effective |
| PHARMACOLOGICAL | ingredient is perillaldehyde. The perillaldehyde and tadeonal |
| EFFECTS | show apparently cooperative effect in anti-fungal effects. |
| | (Kang R. et al. CA, 1912, 117: 2087762) The perillaldehyde |
| | and tadeonal show anti-fungal effects on Trichophyton |
| | rubrum, Trichophyton mentagrophytes, Microsprum |
| | gypseum, Microsprum canis. (Pharmacognology (Japan), |
| | 1984, 38(1): 127). The oil from leaves of Perilla frutescens |
| | shows apparently inhibitive effects on Aspergillus niger, |
| | Penicillium and yeast. (C-Y, Chang et al., China Chinese |
| | medicine, 1990, 15(2): 31) The decoctant of Perilla |
| | frutescens (1:20) show inhibitive effect on ECHO11 viruses |
| | in vitro. (New medicine, 1973,(12): 38) |

NO: 8 Emulsifying oil agents

| | | | |
|---|---|---|---|
| NAME | Guanhuoxiang | OFFICIAL NAME | Pogostemon Cablin |
| | | | (Blanco) Benth |

| | |
|---|---|
| REFERENCE BASIS | China Pharmacopoeia Volume VII pp 130 (China |
| | Sheng-Ghai scientific technology publication) |
| GROWING DISTRICT | Pogostemon Cablin grows in Kwang-tong, Hai-nan and |
| & COLLECT SEASON | Taiwan. Harvest them in August to November or divided |
| & PARTS | into several times. Spread it under the sun till the leaves |
| | shrink then transfer to interior and stack in layers. Put hay |
| | and woods on it and press firmly. Let it dripping for one |
| | night then the twigs and leaves are getting yellow. Spread it |
| | under the sun in next day. Repeat these procedures until |
| | complete dry reached. |
| PREPARATION & | Take 12 kg of Pogostemon Cablin and pressed firmly in the |
| EXTRACTION | distillation flask. By using distillation method to get volatile |
| PROCESS & METHOD | oil. Collect and store them for later use. |
| EFFECTIVE | The stems and leaves of Pogostemon Cablin contain volatile |
| INGREDIENT | oil. Its mainly pathchoulo alcohol, pathchoulo diol, α- |
| | quaiene, δ - quaiene, α -bulnesene, α -pogostene and |
| | pogostone. It also contains β - quaiene, β -elemene, β- |
| | caryophllene, δ -cadinene, and patchoulodiol. |
| ORIGINAL | The pogostone show apparently inhibitive effects on fungi |
| RECORDED | such as C. albicans, Cryptococcus neoformans and Rhizopus |
| PHARMACOLOGICAL | stolonifer. It shows certain degree of inhibitive effect on type |
| EFFECTS | A hemolytic Streptococcus. (J-S, Yang, Science report, 1977, |
| | 22(7): 318) The pogostone can inhibit the growth of fungi |
| | such as Penicillum. (T-S, Kung, Chinese herbs, 1984, |
| | 15(3): 16) |

NO: 9

| | | | |
|---|---|---|---|
| NAME | Tanxiang | OFFICIAL NAME | Santalum album L. |

| | |
|---|---|
| REFERENCE BASIS | China Pharmacopoeia Volume II pp 592 (China Sheng-Ghai |
| | scientific technology publication) |
| GROWING DISTRICT | Santalum album is imported from India, Indonesia and |
| & COLLECT SEASON | Malaysia. |
| & PARTS | |
| PREPARATION & | Take natural yellow colored old blocks of Santalum album |
| EXTRACTION | from the market. Shave them into small pieces or chop them |
| PROCESS & METHOD | into crude powder and pressed firmly in the distillation flask. |
| | By using distillation method to get volatile oil. Collect and |
| | store them for later use. |
| EFFECTIVE | The core wood of Santalum album contains volatile oil |
| INGREDIENT | which is called white sandalwood oil. It's content is about |
| | 1.5% to 5%. The main ingredients are sesquiterpene |
| | compounds such as α - santol and β -santol. |
| ORIGINAL | The α - santol and β -santol show stronger anti-bacteria |
| RECORDED | effects. The bacteria inhibition concentration of santol in |
| PHARMACOLOGICAL | vitro is 1:64000 to 1:128000. It is also effective to Shigella |
| EFFECTS | species and shows inhibitive effect on M. avium at |
| | concentration 1:32000. (Okazakik, et al. CA. 1953, |
| | 47: 7167 g) |

1. A Chinese medicine composition for environmental sanitation comprising Akebia trifoliata, Polygonum perifoliatum, Euodia rutaecarpa, Stemona tuberose, and Bryophyllum pinnatum, and emulsifying agent in which aforesaid elements are dissolved:
wherein an environmental sanitation agent in every 3000 ml of percolate in component proportion comprising Chinese medicine powders of Akebia trifoliata 80-120 gm, Stemona tuberose 80-120 gm, Polygonum perifoliatum 60-100 gm, Bryophyllum pinnatum 50-90 gm, and Euodia rutaecarpa 60-100 gm, and an emulsifying agent in which said Chinese medicine powers are dissolved.

2. The Chinese medicine composition as defined in claim 1, wherein said emulsifying agent comprises oil extracted from Perilla frutescens, Pogostemon Cablin-, and Santalum album-.

3. The Chinese medicine composition as defined in claim 2 further comprising pesticidal Sophora flavescens as adjuvant.

4 (canceled)

5. The environmental sanitation agent as defined in claim 1, wherein said percolate is prepared by liquids comprising distilled water 60-80%, alcohol 12-24%, and glycerin 8-12%.

6. The environmental sanitation agent as defined in claim 1, wherein said emulsifying agent comprises gum Arabic of 30-50 gm, and oil extracted from Perilla frutescens of 30-45 ml, Pogostemon Cablin of 20-35 ml, and Santalum album of 15-25 ml.

7. A for process of preparing an environmental sanitation agent, comprising steps of preparing and dousing Chinese medicine powders of Akebia trifoliata, Polygonum perifoliatum, Euodia rutaecarpa, Stemona tuberose, and Bryophyllum pinnatum into percolate for percolation and then emulsification by an emulsifying agent:
wherein the manufacturing process as defined in claim 9, wherein said percolation comprising steps of percolating said Chinese medicine powders in the percolate of 1200 ml, processing it by a golden mill for 2-3 times, and then dousing it for 2 hours to prepare a first percolate: percolating said Chinese medicine powders in the percolate of 1000 ml and then dousing it for 2 hours to prepare a second percolate: percolating said Chinese medicine powders in distilled water of 1000 ml and then dousing it for 4 hours to prepare a third percolate.

8. The process as defined in claim 7, wherein said environmental sanitation agent in every 3000 ml of percolate in component proportions comprises Chinese medicine powders of Akebia trifoliata of 80-120 gm, Stemona tuberose of 80-120 gm, Polygonum perifoliatum of 60-100 gm, Bryophyllum pinnatum of 50-90 gm, and Euodia rutaecarpa of 60-100 gm.

9. The process as defined in claim 8, wherein said emulsifying agent comprises gum Arabic of 30-50 gm, oil extracted from Perilla frutescens of 30-45 ml, Pogostemon Cablin of 20-35 ml-, and Santalum album of 15-25 ml.

10 (canceled)

11. The process as defined in claim 7, wherein said emulsification further comprises steps of percolation and milling by said golden mill for 3 times.

12. The process as defined in claim 7 further comprising steps of admixture of Mirabilite after said emulsification.

13. The process as defined in claim 11, further comprising steps of admixture of Mirabilite after said emulsification.

14. The process as defined in claim 13, wherein said admixture of Mirabilite comprises steps of adding Mirabilite and benzoic acid and other adjuvents, stirring it evenly, milling it by said golden mill for 2-3 times.
